(19) United States
(12) Patent Application Publication
(10) Pub. No.: US 2017/0347899 A1
(43) Pub. Date: Dec. 7, 2017

(54) METHOD AND SYSTEM FOR CONTINUOUS MONITORING OF CARDIOVASCULAR HEALTH

(71) Applicant: FOURTH FRONTIER TECHNOLOGIES, Pvt. Ltd., Bangalore (IN)

(72) Inventors: MANAV BHUSHAN, BANGALORE (IN); SANDEEP SIBAL, BANGALORE (IN)

(73) Assignee: FOURTH FRONTIER TECHNOLOGIES, Pvt. Ltd., Bangalore (IN)

(21) Appl. No.: 15/465,069

(22) Filed: Mar. 21, 2017

(30) Foreign Application Priority Data
Jun. 3, 2016 (IN) 201641019151
Jun. 3, 2016 (IN) 201641019154
Jun. 3, 2016 (IN) 201641019157
Jun. 3, 2016 (IN) 201641019197

(51) Int. Cl. A61B 5/0205; A61B 5/0402; A61B 8/00; A61B 5/0452; A61B 5/00; A61B 5/053; A61B 5/0456; G06F 19/00; A61B 5/11; A61B 5/08; A61B 5/021; A61B 5/024

(52) U.S. Cl. CPC A61B 5/02055; G06F 19/3418; G06F 19/3406; A61B 5/0402; A61B 8/42; A61B 5/0452; A61B 5/11; A61B 5/0022; A61B 5/0531; A61B 5/0456; A61B 5/7267; A61B 5/021; A61B 5/02427; A61B 2560/0214; A61B 5/08

(57) ABSTRACT

The various embodiments of the present invention provide a system and method for a fully mobile, non-invasive, continuous system for monitoring the cardiovascular health of an individual. The system includes one or more wearable devices affixed on a user, coupled with an application running on a computing device smartphone/tablet, which is connected to a web server in a cloud, and performs various computations on the wearable device, or a smartphone/smartwatch, or the cloud, and provide the user or the concerned personnel with various insights about the general health of the user. The cardiovascular health monitoring system further enables the user to make online appointments, pay online for such appointments, share data with the concerned personnel in a secure manner, and obtain advice and prescriptions through audio/video/text channels.

### CROSS-REFERENCE TO RELATED APPLICATIONS

This application claims priority and benefit of Indian patent applications:

2016/41019151 filed Jun. 3, 2016;

2016/41019197 filed Jun. 3, 2016;

2016/41019154 filed Jun. 3, 2016; and

2016/41019157 filed Jun. 3, 2016. This application is also related to U.S. non-provisional patent application Ser. No. 15/373,735 filed Dec. 9, 2016. The disclosures of the above provisional and non-provisional patent applications are hereby incorporated herein by reference.

### BACKGROUND

### Field of the Invention

The present invention is generally related to health monitoring devices. The present invention is particularly related to a device and system for monitoring cardiovascular health and analysing healthcare data. The present invention is more particularly related to cardiovascular health monitoring, and analysis of haemodynamic parameters using wearable devices.

### SUMMARY

To effectively circumvent this shortage of medical personnel and infrastructure, technological solutions that allow monitoring and treatment of patients without forcing them to come to a clinic or hospital physically for each step of the treatment, with minimal time involvement of medical practitioners, to make the entire system more efficient, would be helpful.

The various embodiments of the present invention provide a system and method for continuous health monitoring of the User, with a particular focus on cardiovascular health. The system includes a wearable device that is coupled with a disposable two-sided sticker, which together form a patch called a ‘Biostrip’ device. The Biostrip device includes a plurality of electrodes, an electronic circuitry to measure electric potentials for one or more channels, and/or a circuitry for measuring electrical impedance on the skin using electrodes, and/or one or more accelerometers, and/or a reflectance-based Photoplethysmograph (PPG) sensor module. The wearable device is designed to measure the electrocardiogram (ECG) and/or heart rate and/or respiration cycles and/or blood oxygenation and/or the seismocardiography (SCG) and/or body movements and/or blood pressure (BP). The wearable device may also include a blood glucose sensor, and/or a sensor to measure levels of Haemoglobin (Hb) and other blood gases (such as Carbon Dioxide) of the user.

Embodiments of the present invention include a health monitoring system that optionally allows the user to send the medical data securely to another individual or institution. Further, the present invention optionally includes a system and method for initiating appointments and conduct online consultations with Physicians/other medical experts when required. The system monitors a plurality of health parameters including, but not limited to: Electrocardiography (ECG), Seismocardiography (SCG), Skin Impedance, Electrodermal Activity (EDA), Skin Temperature, Blood Oxygenation, CO₂ levels in the blood, Haemoglobin levels in the blood, Blood Glucose and Blood Pressure. According to an embodiment of the present invention, the system has the ability to record medical data from a plurality of Biostrip devices worn by multiple Users, to record the data to a secure location in the Cloud, and touse techniques such as machine learning, artificial intelligence to derive insights and provide recommendations for each particular user, and to facilitate online Consultations with data sharing.

The various embodiments of the present invention provide systems and methods for monitoring and analysing biosignals measured by one or more wearable devices (Biostrips), and alerting the user and the concerned personnel when certain conditions are detected.

These and other aspects of the embodiments herein will be better appreciated and understood when considered in conjunction with the following description and the accompanying drawings. It should be understood, however, that the following descriptions, while indicating the preferred embodiments and numerous specific details thereof, are given by way of an illustration and not of a limitation. Many changes and modifications may be made within the scope of the embodiments herein without departing from the spirit thereof, and the embodiments herein include all such modifications.

The other objects, features, and advantages will occur to those skilled in the art from the following description of the preferred embodiment and the accompanying drawings.

### BRIEF DESCRIPTION OF THE DRAWINGS

FIG. 1 shows a schematic layout of the Biostrip Device (115) and shows a plurality of sensors (101, 102, 103, 104), that record data, and send the data to a microcontroller or microprocessor or another computing device (105) on the Biostrip.

FIG. 2 shows a picture of the internal components of the Biostrip (115), in the actual configuration, as used in the product, according to various embodiments.

FIG. 3 shows a process flowchart for how the Biostrip (115) and accompanying application function, in various embodiments.

FIG. 4 shows a picture of the Biostrip (115) coupled with the double-sided sticker (114), one side of which adheres to the Biostrip (115), and the other side adheres to the skin of a User, when the Biostrip (115) is worn by a User. The sticker (114) also contains cut-out holes for the sensors (101,103), so that they can be in direct contact with the skin.

FIG. 5 shows a picture of the Biostrip device (115) worn by a User, in various embodiments, stuck on the User's sternum.

FIG. 6 shows the underside of the encased Biostrip (115), showing some of the sensors (101, 103) and other components, which together make up the Biostrip device (115).

FIG. 7 shows various embodiments of the health monitoring system, comprising of the Biostrip device (115), which sends the data collected from the User to the smartphone or gateway device (116). The smartphone (116) then sends the data to the web server (117), where the data is processed, stored and/or analyzed in greater detail. Thereafter, the system can send alerts to the User, caregiver and/or Doctor, in various embodiments.

FIG. 8 illustrates various embodiments of the process of monitoring a single patient.

FIG. 9 shows a block diagram for the collection of physiological data by multiple Biostrips for multiple Users (125), in various embodiments.

FIG. 10 shows an example of the signals captured on the Biostrip device (115), comprising of the ECG signal (132) and the SCG signal (133), which together denote several events of the cardiac cycle.

### DETAILED DESCRIPTION

In the following detailed description, a reference is made to the accompanying drawings that form a part hereof, and in which the specific embodiments that may be practiced is shown by way of illustration. These embodiments are described in sufficient detail to enable those skilled in the art to practice the embodiments and it is to be understood that the logical, mechanical and other changes may be made without departing from the scope of the embodiments. The following detailed description is therefore not to be taken in a limiting sense.

The various embodiments of the present invention provide a system and method for monitoring the health of a user continuously. The system comprises of one or more wearable devices (Biostrips) which communicate wirelessly to a gateway device such as a smartphone/smartwatch/router. The Biostrip device comprises an electronic module or a component that is reusable and rechargeable (via any wire, such as a micro-USB/firewire/Pogo pins or wirelessly or both) or disposable and non-rechargeable, and is stuck on one side with a two-sided adhesive tape, which adheres to the skin of the User on the other side.

In various embodiments, the Biostrip device is fabricated upon a flexible printed circuit board (PCB), or on two or more hard PCBs connected with flexible PCBs (together making up a rigid-flex PCB), or on any combination of flexible or hard PCBs. According to an embodiment of the present invention, the width of the wearable device is 5-150 mm in length, 3-150 mm in width, and 1-50 mm in height, and at least part of the Biostrip device is flexible, and adapts to almost any surface on the body including the forehead or abdomen or chest of the user.

In various embodiments, the Biostrip device includes a vibration motor to alert the user under certain pre-defined circumstances. Alerts are sent when some abnormality is detected from the bio-signals being recorded—either as computed on a Multipoint Control Unit (MCU) itself in real-time, or as computed on the web server on the cloud and then communicated to the Biostrip device by way of Bluetooth of some other wireless communication protocol, or according to the findings of a doctor looking at the database on the web client communicated to the Biostrip by way of Bluetooth of some other wireless communication protocol.

In various embodiments, the Biostrip device includes one or more LEDs, visible through the casing, or placed on top of the casing, which communicates different information about the device status and functionality to the user, and/or a microprocessor or other processor to collect data from the multiple sensors, and perform different kinds of algorithms on the wearable device itself.

In various embodiments, the Biostrip device includes an integrated circuit (IC) for wireless data communication, that enables it to connect and communicate and send and receive data from a smartphone/smartwatch or another gateway device. Further, the Biostrip device may include a memory chip that allows it to store data for long periods of time, and then to communicate this saved data to other locations.

In various embodiments, the Biostrip device contains an audio speaker that allows the User to hear certain alerts or audio commands. The Biostrip device may also contain an audio recorder that allows the User to record or send audio instructions to the Biostrip.

In various embodiments, the wearable device may be re-charged through a wired connection, such as a micro-USB connection/firewire/pogo pin, or through a wireless charger, and hence can be reused many times. Further, the Biostrip device may have a casing which is waterproof, and may therefore be used in conditions where there are water and rain, or under water.

In various embodiments, the Biostrip device includes a reflective Photoplethysmograph (PPG) module attached to the underside of the device, and in direct visual contact with the skin on the chest/wrist/forehead or other location where the device adheres. The PPG module comprises of two or more light emitting diodes (LEDs), and one or more photodiodes, which measure the changes in the intensity of reflected light of one or more wavelengths. The PPG module may be capable of measuring blood oxygenation, and/or levels of Haemoglobin, and/or other blood gases, such as carbon dioxide (CO₂), and/or heart rate, and/or other measures derived from changes in blood flow. In various embodiments, the device uses the above-mentioned optical sensor, or a different sensor, emitting Electromagnetic waves at two or more wavelengths, to measure Blood Glucose levels.

In various embodiments, the Biostrip comprises of the following elements: two or more electrodes connected to a single analog front end (AFE) system, which may further transmit the signal to an analog-to-digital converter (ADC) and then on to an MCU. The Biostrip device may also contain a digital PPG sensor and/or one or more accelerometers and/or a temperature sensor configured for measuring the skin temperature, at the location where the device is affixed to the body.

In various embodiments, the electrodes, AFE and MCU together measure and record electrical signals comprising of the Electrocardiogram (ECG) when stuck on the chest, or Electroencephalogram (EEG) when stuck on the forehead, or electromyogram (EMG) when stuck on a muscle, or a combination of all three signals.

In various embodiments, the Biostrip device measures inhalation and exhalation cycles using an electrical impedance measured between two or more electrodes, and/or from the movements of the accelerometer(s), and/or from the signal measured on the PPG module, and/or from the variation in the magnitude of the R-peaks as measured on the ECG sensor.

In various embodiments, the Biostrip device includes one or more accelerometers capable of measuring acceleration within a range of 0.01 milliG-20 G, and hence capable of measuring steps, breathing, heartbeats, opening and closing of heartvalves (the aortic and mitral valves), rapid ejection and rapid filling, when placed at different locations on the body. The accelerometer(s) record Seismocardiography (SCG) when affixed to particular parts of the User's chest. The accelerometers may also include a tap-detection functionality, which allow the user to activate different kinds of processes with a single/double tap.

In various embodiments, the Biostrip device, when affixed vertically or horizontally on the sternum, or any other location on the chest, uses the SCG data collected from the accelerometer, to detect cardiac events including, but not limited to: Heart murmurs, Aortic valve opening (AO), Mitral valve opening (MO), Aortic valve closure (AC), Mitral valve closure (MC), Rapid Ejection (RE), Rapid Filling (RF), and Atrial Systole (AS), the peak after the AO event on the y-axis of the SCG (J-wave).

In various embodiments, the Biostrip device combines the information from the ECG and/or the PPG and/or the SCG signals, to calculate a value of Pulse Transit Time (PTT) from the R-peak of the ECG or the aortic valve opening (AO) peak of the SCG, till the foot/peak/dichrotic notch of the PPG curve, which is then used to calculate measures of Systolic and Diastolic Blood Pressure of the person wearing the device, corresponding to each heart-beat recorded on the device. The Blood Pressure may be computed after calibrating for each User against a regular sphygmomanometer, or it may be computed by using a Regression model estimated from a population database.

In various embodiments, the User can wear one or more Biostrip devices, which connect with a single gateway device such as a smartphone/smartwatch/router wirelessly, using a wireless communication protocol such as Bluetooth or Wifi. The Biostrip devices first sync their internal clocks with the real time clock (RTC) of the gateway device—smartphone/smartwatch, other gateway device—so that the internal clocks of the Biostrips are aligned with the clock of the gateway device, as well as with each other. This synchronization is achieved by the smartphone sending its exact UTC time to the Biostrips via Bluetooth, or some other wireless communication protocol, and the Biostrips then updating their RTC to this exact time (to a resolution of milliseconds), plus a delta which has been calculated previously, and which is the time delay between the sending of the Bluetooth Low Energy (BLE) packet by the smartphone, and the updating of the RTC on the Biostrip. This process of synchronization is optionally repeated every 1 hour or as needed, so that any drift between the clocks of different Biostrips is normalized every 1 hour.

In various embodiments, in order to synchronize the clocks of the Biostrips with the smartphone, the time delay between the sending of the BLE packet by the smartphone, and the updating of the RTC by the Biostrip, is calculated by first sending the current time from the smartphone to the Biostrip (say t₁), then updating the RTC on the Biostrip to time t₁, and then immediately sending another BLE packet from the Biostrip back to the smartphone, with the latest time of the Biostrip (say t₂). If this packet is received by the smartphone at time t₃, then it is assumed that the correction factor (delta) to be added to the RTC on the Biostrip is: (t₃−t₁)/2.

In various embodiments, the RTC of the smartphone is synced with one or more Biostrips by putting the Biostrip and the smartphone on the same table, next to each other, and then sharply tapping the table with your fingers at intervals of 1-6 seconds or more. The tapping procedure is repeated one or more times, and the sharp spike in the accelerometer recordings of the Biostrip and the smartphone are used to determine the time delay between the RTC of the smartphone and the Biostrip. This is done by algorithms running on the Biostrip and the smartphone, which calculate the time of the sharp peak in the accelerometer data caused by the tap, with a resolution of 1 millisecond or less. If the timing of the peak is calculated to be t₁ on the smartphone, and t₂ on the Biostrip, then the RTC of the Biostrip is updated by adding a correction factor equal to (t₁-t₂) to the RTC of the Biostrip.

In various embodiments, the internal clocks of two or more Biostrips are combined with each other, by wearing the multiple Biostrips on different locations of the chest, or wearing one or more on the chest and holding one Biostrip, with one finger or each hand on the right and left electrodes, at the same time. In this configuration, the algorithms running on the Biostrips calculate the timing of the R-peak on the ECG with a resolution of less than 1 millisecond, and then the difference between the timings of the R-peak on the different Biostrips is used as a measure of the delay between the different Biostrips, and the RTCs of the Biostrips are updated by adding these differences to their respective RTCs.

In various embodiments, the User wears one Biostrip on the chest, which measures ECG and SCG, and wears another Biostrip on the forehead or wrist or foot, or some other location where the microvasculature is close to the skin. In these embodiments, the internal clocks of the two Biostrips are aligned as described herein, and the Pulse-Transit Time (PTT) is estimated by calculating the time delay between the R-peak or the AO-peak measured on the Biostrip located on the chest, and the peak and foot of the PPG curve measured on the second Biostrip located on the forehead/wrist/foot. This is used to calculate a value for peripheral systolic and diastolic Blood Pressure.

In various embodiments, the Biostrip(s) transfer data to the smartphone/smartwatch or other gateway device wirelessly via Bluetooth or some other near field communication (NFC) protocol, and then from there the data is transferred to a web client via Wi-Fi, and stored in a secure database on a web server. In another embodiment, the data is transferred directly from the Biostrip to the web using Wi-Fi or 3G/4G wireless communication. This data can then be accessed by doctors or caregivers or the User themselves, using a web application, and historical data for each patient can be viewed and analysed.

In various embodiments, the data stored on the web for multiple Users, is used in Machine learning algorithms such as a convolutional neural networks and/or Bayesian Classifiers and/or support vector machines, to distinguish between healthy and pathological conditions of the User in question, by using the stored and annotated data as a training set, and applying the classification algorithms on the User's data.

In various embodiments, the system also allows the User or the caregiver to book appointments with a concerned personnel at a given hospital or clinic, and also to conduct an ‘Online Consultation’, by way of an audio/video VOIP call, or a regular phone call, or over a wireless communication protocol over the web, using the smartphone application that collects data from the Biostrip. In this protocol, the User can also share his/her data with the Doctor or care-giver with whom the online consultation has been scheduled. During an online consultation, the system also allows the doctor or the concerned medical practitioner to view certain parts of the data generated by the wearable device, and to make episodic measurements from the Biostrip device, through instructions delivered to the patient, and thereafter to view the data in near real-time through a web application. In this manner, the system facilitates a ‘Virtual Examination’.

In various embodiments, the system uses information collected from other sensors on the computing device such as a smartphone or a tablet or other device, about the environment of the user, in order to present the parent/caregiver/doctor with daily/weekly/monthly report of the general health parameters recorded during different activities, at different times of the day. The application on the phone also provides recommendations to the user or caregiver or doctor for different ways of improving the health parameters at different times of the day.

In various embodiments, the system stores the individual parameters, including but not limited to age, sex, medical history, for each user, and additionally combines anonymised information of multiple users from different locations on the cloud, in order to present each individual user with data about the health parameters of the user and activities in comparison with a similar population, and in order to give the user information about where they stand with respect to other users, in terms of percentiles and other comparative measures. Further, the system combines subjective information and information from multiple sensors on multiple users, as described above, to run Machine Learning algorithms, Bayesian classifiers or other kinds of training and testing protocols, in order to provide alerts that are customized for each individual, based on the normal parameters for the users in the same demographic section.

According to some embodiments of the present invention, 2-6 Biostrip devices are used together on a single subject, stuck at different locations on the chest, to mimic a 2-6 Lead Holter monitor, recording and transmitting ECG for cardiac patients. The clocks of the multiple Biostrips are aligned with each other, as described herein. According to these embodiments, the system automatically detects cardiovascular abnormalities including, but not limited to: tachycardia, brachycardia, arrhythmia (of the different kinds): from the single-lead ECG, or a combination of 2-6 Biostrips mimicking a 2-6 lead Holter monitor, as described herein.

According to some embodiments of the present invention, the Biostrip device also includes an Ultrasound transducer, which produces Ultrasound signals in the range of 0.5-30 KHz, which reflect off the heart walls, when the device is placed on the chest, close to the heart, and then receives the back-propagated echoes. This Ultrasound measurement is capable of being triggered by a command from the application running on the smartphone, for a duration of 0.1-10 mins, or of being triggered periodically by a timer on the Biostrip device or the smartphone application, to enable periodic measurements.

According to other embodiments of the present invention, a Biostrip device that contains an Ultrasound transducer as described herein is used to measure the Stroke Volume, Cardiac Output, Pre-ejection period (PEP), Left Ventricular Ejection time (LVET), the E/A ratio and other quantities that are usually measured in an echocardiogram, when stuck on the left part of the chest, close to the heart. According to these embodiments of the present invention, a Biostrip device that includes an Ultrasound transducer as described herein, is stuck on the sternum, and used to measure the width and thickness of the aorta, which is then used in the Bramwell-Hill equation to get a more accurate measure of the Aortic pulse wave velocity, calculated by first estimating the pulse transit time from the opening of the aortic valve (AO) till the time-point at which the pressure pulse wave hits the aortic arch, and then normalizing with the length, width and thickness of the aorta until the aortic arch, as measured by the Ultrasound transducer on board the device.

In various embodiments, the Biostrip device records the ECG data at a frequency of anywhere between 125 Hz and 4 kHz, PPG data at a frequency of anywhere between 25 Hz and 2 kHz, Accelerometer data at a frequency of anywhere between 5 Hz and 2 kHz, and sends the data to the MCU, where the data is processed using mean/median/Bandpass filters, and automated peak detection algorithms annotate each signal, and calculate the timing of the electrical, mechanical and blood-flow related events in the cardiac cycle.

In various embodiments, the pre-ejection period (PEP) is measured indirectly by calculating the time interval between the R-wave on the ECG and the J-wave on the y-axis of the accelerometer (R-Jᵢₙₜₑᵣᵥₐₗ). The equation used to derive PEP is of the form:

PEP=x₁*R-Jᵢₙₜₑᵣᵥₐₗ;
where the constant x₁ determined independently from his/her data, or from a population database.

In various embodiments, the Biostrip device records the ECG data as described above, and uses an algorithm running on the MCU, which uses the 3-10 data points around the P/Q/R/S/T peak, and then uses wavelet transforms or other peak-interpolation techniques to further improve the accuracy of the detected peaks to less than 1 millisecond.

In various embodiments, the Biostrip device collects PPG data corresponding to one or more wavelengths, from any location on the body which has micro-vasculature, and uses the data collected to estimate quantities including, but not limited to: PPG amplitude, time of foot of the pulse wave, peak of pulse wave, timing of dichrotic notch, timing of 25% mark (the time at which the PPG pulse wave achieves 25% of its maximum width/height), timing of 50% mark, timing of 75% mark, width of pulse wave at 25%, 50% and/or 75% marks, and the like. The PPG data for two or more wavelengths is used to estimate the peripheral Oxygen saturation in the blood (SpO₂), which is expressed as a percentage.

According to various embodiments, the Biostrip device uses data from the ECG sensor, PPG sensor, and the SCG as measured from the accelerometer, as described herein, and when the Biostrip device is affixed on some part of the chest, to calculate values for the cardiac time intervals (CTIs) including, but not limited to: Pre-ejection period (PEP), left ventricular ejection time (LVET), Q-wave of ECG to the first sound from the Phonocardiogram (QS1), Q-wave of ECG to the second sound from the Phonocardiogram (QS2), first sound to second sound of Phonocardiogram (S1S2), PR-interval from ECG, QRS duration from ECG, the time interval between the R-wave on the ECG, the J-wave on the y-axis of the SCG (R-J interval), Systolic Time, Diastolic Time, PTT_{foot}, PTTₚₑₐₖ, Electro-mechanical Activation time (R-peak to MC), Isovolumetric Relaxation Time (IVRT), which is the time interval from AC to MO on the Seismocardiogram, and Isovolumetric Contraction Time (IVCT), which is the time interval from MC to AO, and/or the like.

In various embodiments of the system, the values of PEP and LVET as calculated from the Biostrip device, as described elsewhere herein, are used to calculate a beat-to-beat value of PEP, LVET and PEP/LVET, which is combined with values of instantaneous heart rate (IHR), as measured by ECG, Pulse Wave Velocity (PWV), as measured from ECG and PPG, amplitude of PPG peak, amplitude of AO peak, body mass index (BMI), to arrive at a measure of a Cardiac Health Index (CHI), and/or the like. In other words, we derive a function:

CHI=x₁*PEP+x₂*LVET+x₃*(PEP/LVET)+x₄*amp(AO)+x₅*amp(PPG)+x₆*IHR+x₇*PWV; or

CHI=ƒ(PEP,LVET,R-Jᵢₙₜₑᵣᵥₐₗ,amp(AO),amp(J-wave),amp(PPG),IHR,PWV,SpO₂,height,weight,BMI,age,blood profile,genetic profile);

Here, Where the constants xᵢ are calculated as a weighted average of the User's age, Body Mass Index (BMI), height, weight and other medical history and Blood lipid profile (if available) and gender, and ƒ is a linear or non-linear function that maps the input parameters to the output variable CHI.

In various embodiments of the invention, the values described herein are used to calculate the Tei index, or the Myocardical Performance Index, as MPI=(IVRT+IVCT)/LVET. This value is calculated for some or every heartbeat on the Biostrip device, and is sent to the accompanying smartphone or other gateway device via Bluetooth, or some other NFC protocol.

In other embodiments of the invention, a quantity called the Myocardial Exercise Performance Index (MEPI) is calculated. For example, the Biostrip device may be configured to calculate a change in MPI per unit power (measured in kilo-Joules per second), i.e. the change in MPI as the subject undergoes exercise. The amount of exercise is measured by the second accelerometer on the Biostrip device, when it is sampling at a rate of 50-800 Hz, in the setting of +/−8 g, while the first accelerometer, measuring SCG, is set to record at 100-800 Hz in the +/−2 g setting. The recording of the second accelerometer is converted to Energy using the weight of the individual, and by integrating (summing over) time to arrive at:

E=F·S=mass×acc×dist,
where acc is measured by combining the acceleration vector across each of the three axes, and the distance S is measured by:

S=ut+½*a*t*t,
where the calculations are made for periods where the subject is starting from rest.
Using the above relationships, MPI is calculated when the person is first at rest (at time t₁), and then calculated again at time t₂, after the person has undergone exercise for a period of time (t₂−t₁). Thus, the MEPI is given by:

MEPI={MPI(t₂)−MPI(t₁)}/{F·S/(t₂−t₁)}

In other embodiments of the present invention, the procedure described herein to calculate a measure of exercise, is used to calculate a change in the Cardiac Health Index (CHI) as described above, to arrive at a new quantity known as the Cardiac Exercise Health Index (CEHI), which is first measured at when the person is first at rest (at time t₁), and then calculated again at time t₂, after the person has undergone exercise for a period of time (t₂−t₁). Thus, the CEHI is given by:

CEHI={CHI(t₂)−CHI(t₁)}/{F·S(t₂−t₁)}.

According to another embodiment of the present invention, the Cardiac Time Intervals described herein are used to calculate a value for Left Ventricular Ejection fraction (LVEF) or Ejection Fraction (EF) in the form of:

LVEF=y₁*PEP+y₂*LVET+y₃*(PEP/LVET)+y₄*amp(AO)+y₅*amp(PPG)+y₆*HR+y₇*PWV+y₈*IVRT+y₉*IVCT; or

LVEF=ƒ(PEP,LVET,R-Jᵢₙₜₑᵣᵥₐₗ,amp(AO),amp(J-wave),amp(PPG),IHR,PWV,IVRT,IVCT,height,weight,age,BMI,blood profile,genetic profile);

Here, the constants yᵢ are derived from a population database, which assigns a distance for the individual being measured, to other subjects in the database, on the basis of height, weight, age, BMI, prior medical history and gender. If other information, such as blood test reports and/or echocardiograms and/or genetic tests are available, then these are also used to calculate the distance of the individual being measured to a similar subgroup (1-100 people) of the population. Once the distance function is derived, the correlation coefficients in the above equation are calculated by taking a weighted average of correlation coefficients previously calculated for patients who were simultaneously measured for LVEF using the Biostrip, and a standard Echocardiograph. Further, ƒ may be any linear or non-linear function which maps the input parameters to the output LVEF.

According to various embodiments of the present invention, the change in LVEF in response to exercise, is measured by combining the method described above, to measure LVEF, along with the method of quantifying exercise, as described herein, and is used to calculated a quantity described hereon as the “Exercise Modulated Ejection Fraction” (EMEF), which is first measured at when the person is first at rest (at time t₁), and then calculated again at time t₂, after the person has undergone exercise for a period of time (t₂−t₁). Thus, the EMEF is given by:

EMEF={EF(t₂)−EF(t₁)}/EF(t₁)*{F·S/(t₂−t₁)}.

According to various embodiments of the present invention, the Cardiac Time Intervals described herein are used to calculate a values for Stroke Volume (SV) and Cardiac Output (CO) in the form of:

SV=y₁*PEP+y₂*LVET+y₃*(PEP/LVET)+y₄*amp(AO)/Avg(AO)+y₅*amp(PPG)/Avg(PPG)+y₇*(1/PTT_{foot})+y₈*(1/PTTₚₑₐₖ)+y₉*(1/PTT_{notch})+y₁₀*(1/PTT_{foot})²+y₁₁*(1/PTTₚₑₐₖ)²+y₁₂*(1/PTT_{notch})²; or

SV=ƒ(PEP,LVET,R-J-interval,amp(AO),amp(J-wave),Avg(AO),amp(PPG),Avg(PPG),PTTₚₑₐₖ,PTT_{foot},PTT_{notch},age,gender,height,weight,BMI); and

CO=SV*IHR;

Here, the constants yᵢ are typically derived from a population database, which assigns a distance for the individual being measured, to other subjects in the database, on the basis of height, weight, age, BMI, prior medical history and gender, and ƒ is a linear or non-linear function. If other information, such as blood test reports and/or echocardiograms and/or genetic information are available, then these are optionally also used to calculate the distance of the individual being measured to a similar subgroup (1-100 people) of the population. PTT_{foot} denotes the Pulse Transit Time between the R-peak from the ECG and the foot (beginning) of the PPG pulse wave, PTTₚₑₐₖ denotes the Pulse Transit Time between the R-peak from the ECG and the peak of the PPG pulse wave, PTT_{notch} denotes the Pulse Transit Time between the R-peak from the ECG and the dichrotic notch of the PPG pulse wave, and IHR denotes the instantaneous Heart Rate. Once the distance function is derived, the correlation coefficients in the above equation are calculated by taking a weighted average of correlation coefficients previously calculated for patients who were simultaneously measured for SV using the Biostrip, and a standard Echocardiograph.

In another embodiment of the present invention, the Exercise Modulated Stroke Volume (EMSV) and Exercise Modulated Cardiac Output (EMCO) are calculated in exactly the same way as described herein, by normalising with the amount of exercise in KJ per second.

In another embodiment of the present invention, the response to exercise in EF, SV, CO, MPI and CHI are measured by making the patient undergo a particular exercise regimen, from a choice of: (a) cycling on an exercise bike for 2 mins, at a speed of 15 kmph; (b) cycling on an exercise bike for 4 mins, at a speed of 10 kmph; (c) running on a treadmill for 2 mins, at a speed of 10 kmph, and an incline of 10 degrees; (d) running on a treadmill for 4 mins, at a speed of 10 kmph, and an incline of 10 degrees; and/or the like. In this case, EF, SV, CO, MPI and CHI are measured as described above, before and after the chosen exercise, and the absolute and relative response in the particular quantity (ΔQ) are measured as:

ΔQ_{abs}=Q_{after}−Q_{before};

ΔQᵣₑₗ=(Q_{after}−Q_{before})/Q_{before};

Here each measured value is calculated specifically for the quantity (EF, SV, CO, MPI or CHI) and the exercise regimen in question.

In various embodiments, the values for Exercise Modulated EF, SV, CO, MPI and CHI, and response in EF, SV, CO, MPI and CHI to exercise, are used to calculate a value of “Cardiac Health Risk” (CHR), which gives the percentage probability of a Major Cardiovascular Event (MACE) in the next 1 year. This value is coupled with a values for the confidence interval of each measurement. If an abnormal (>10%) value of CHR is recorded with a high (>95%) confidence interval, then an automatic alert may be sent to the User via the vibration motor on the device, and further alerts may be sent to the Doctor or care-giver registered by the User, on the application on the smartphone.

In various embodiments of the present invention, in addition to calculating LVEF, MPI, CHI as mentioned herein, the system computes a value of the LVEF, Amp(AO), Amp(RF) and PEP measured before and after a pre-specified exercise regimen, to calculate a value of “Cardiac Health Risk” (CHR), which gives the percentage probability of a Major Cardiovascular Event (MACE) in the next 1 year. CHR is calculated in the following manner:

CHR=x₁*{(Amp(AO_{before})−Amp(AO_{after}))/Amp(AO_{before})}+x₂*{(Amp(RF_{after})−Amp(RF_{before}))/Amp(RF_{before})}+x₃*(PEP_{after}−PEP_{before})/PEP_{before}+x₄*(LVEF_{before}−LVEF_{after})/LVEF_{before}; or

CHR=ƒ(Amp(AO_{before},Amp(AO_{after}),Amp(J-wave_{before}),Amp(J-wave_{after}),Amp(RF_{before}),Amp(RF_{after}),PEP_{after},PEP_{before},LVET_{before},LVET_{after},LVEF_{before},LVEF_{after},BMI,height,weight,age,gender,blood profile,genetic profile);

Where the xᵢ are constants that form a probability distribution function, determined for each individual on the basis of their height, weight, gender, BMI, age and prior medical history. Here the value of CHR is given in percentage, and the confidence interval for the value is given by a product of the confidence interval for the measurement of the SCG signal, which is measured in the manner described below, and ƒ is a linear or non-linear function that maps the input variables to the output, CHR.

In various embodiments of the present invention, the signal quality of each individual sensor is optionally assessed and a confidence interval is calculated for signals including ECG, PPG and SCG. In order to assess signal quality, the method of (a) template matching, after normalizing the y-axis values between min and max (i.e. between 0 and 1), and by normalizing the x-axis in such a manner so that at least 2 peaks (R-peaks from ECG, AO-peaks from SCG, Pulse wave peak from PPG) in the window occur at the same positions; (b) comparison of FFTs of the measured and ideal signals; (c) comparison of the Histograms of the measured and ideal signals.

In various embodiments of the system, the processor running on the device first collects data from the sensors, and also performs signal processing and cleaning, using one of the following protocols:
a) Calculate the SSD (sum of squared differences) value of the Histogram of the measured signal and Histogram of the ideal signal X (Xᵢ, after applying a Bandpass filter with a low cut-off of 2 Hz, and a high cut-off of 30 Hz for ECG, cut-offs of 0.01 and 200 Hz for accelerometer, 2 Hz and 40 Hz for PPG, using 20 bins, 2×fₛ samples (where fₛ is the sampling frequency) for the Histogram, and 8×fₛ or 4×fₛ number of samples in each window for the Bandpass filter (e.g. 1000 or 500 sample window for a sampling rate of 125 Hz). The Histogram of the ideal signal Xᵢ is stored in the CPU memory before-hand, to perform this estimation. Signal quality of a 2×fₛ sample window is now calculated to be:

Signal_Quality(X)=max{0,[(20−SSD(X,Xᵢ)/X)/20×100]}
This assumes a threshold of 20, or in other words assumes that a Chi-squared value of more than 20 means that the signal is pure noise. However, this threshold may be automatically adjusted based on other measurements.
b) Calculate the FFT of the measured signal X, after applying Bandpass filters as described above, and calculate the SSD value of the FFT of the measured signal, and FFT of the ideal signal (ECGᵢ). This value: SSD(FFT(X), FFT(Xᵢ))/FFT(X), can be used to quantify signal quality in a manner similar to (a) above.
c) In the method of template matching, a sample of the ideal signal, for 8×fₛ or 4×fₛ number of samples is stored in the local memory, and after the peaks are detected on the signal X (R-peak on ECG, AO-peak on SCG and Pulse wave peak on PPG), the measured signal is re-interpolated using linear interpolation so that the peaks on the measured signal X_{obs} are in exact correspondence with the peaks on the ideal signal Xᵢ. Once Xᵢ and X_{obs} are in exact correspondence according to their peaks, the signals are normalised to values between 0 and 1, and then the SSD is calculated between the two signals. For reference, the value of SSD between Xᵢ and a vector of white Gaussian noise, between 0 and 1, is calculated and stored in the internal memory of the device as SSDₓₙ, and then the final value for signal quality is given as:

Signal Quality=max{1−SSD/SSDₓₙ,0}×100

According to another embodiment of the present invention, the pre-ejection period (PEP) is calculated before and during exercise being conducted by the User, and as the User is exercising, assuming that the User starts from rest at time t₁, the change in PEP due to exercise is calculated as:

ΔPEP=PEPₜ₂−PEPₜ₁

Simultaneously, the power for any period of time Δt=t₂−t₁, is calculated as: P=F·S/Δt. The power (P), and the pre-ejection period (PEP) is calculated for every 10 sec interval, and the change in power (ΔP) and change in PEP (ΔPEP) is calculated between every two successive intervals. Then a value known as the Exercise PEP Index (EPEPI), is calculated as:

EPEPI=ΔP*ΔPEP.

According to various embodiments of the present invention, if the EPEPI index is calculated to be above a certain threshold thresh>0, then an alert is sent to the User through the vibration motor, or the smartphone application. This gives an indication to the User to not exert themselves any further, as the capacity of the heart to increase contractility further is exhausted. The exact value of thresh is calculated separately for each individual, on the basis of his/her age, fitness levels, prior medical history.

In one embodiment of the present invention, the quantities mentioned herein that need only ECG and PPG for their calculation, are calculated while the person holds the Biostrip device with one finger from each hand on the left and right electrodes of the Biostrip, and the tip of another finger touching the PPG sensor, so as to enable the measurement of ECG and PPG from the hands simultaneously. The User is optionally required to hold the device in this configuration for 2-60 secs, while the PQRS complex from the ECG, and the foot, peak, dichrotic notch, 25% mark and 75% mark are identified from the PPG curve.

According to an embodiment of the present invention, a ‘Virtual Cardiac Health Examination’ (VCHE) is carried out when the User is wearing the device on his/her chest. Under this routine, the User first wears the device on the chest, then assumes a supine position, and stays still and breathes slowly, and then gives a command through the smartphone application, to trigger the VCHE, which takes 2-300 seconds. In addition to calculating PEP, LVET, EF, CHI, MPI, Blood Pressure, Heart Rate, the device optionally also estimates the 3D vector corresponding to each event in the SCG cycle (AC, AO, MC, MO, RF, FE). The direction (identified in terms of one of the eight possible octants) and magnitude of these vectors is then compared to the average values for the direction and magnitude of the vectors associated with the same events in the general population, and in the same individual from earlier recordings, to distinguish between healthy states, and states of Ischemia, Myocardial Infarction and Atrial Flutter. In general, this identification is based on the amount of inconsistency between the vectors corresponding to successive measurements of the same cardiac events.

In various embodiments of the system, the time-series data for parameters including but not limited to: ECG, PPG, SCG, skin temperature, exercise (as measured by the accelerometer) and/or movements (as measured by the GPS on the smartphone) are combined on the web server, and together used in a model measuring Granger causality, to give the User or his/her caregiver a view of which parameters affect the cardiovascular health of the subject.

According to some embodiments of the present invention, the Biostrip device and accompanying calculations on a cloud computing system are used for Sleep Apnea detection and treatment.

According to some embodiments of the present invention, the Biostrip device and accompanying calculations on the cloud server are used for detecting and diseases in infants, and for monitoring their health and activities in a continuous and non-invasive manner. Further, the system also optionally includes continuous information about the posture of the infant and an alerting mechanism for fall detection, or detection that the infant is laying and/or sleeping face down.

According to some embodiments of the present invention, the wearable device and accompanying algorithms on the cloud server are used for monitoring the health and activities of an elderly patient, by their relatives or caregivers situated in a remote location. In this case, the system may also include information about the location, movements, posture of the subject, as well as an alerting mechanism for fall-detection.

According to some embodiments of the present invention, a computational system comprising of a Convolutional Neural Network, with a pre-loaded set of clean and noisy signal samples, is used for the purpose denoising the ECG, PPG and SCG signals. The computational system runs on the Biostrip device, or the Smartphone application, or the server in the cloud, and includes a database of clean and noisy signal samples of each individual signal, collected from the Biostrip, and stored in the memory of the Biostrip/smartphone app/Cloud server.

According to some embodiments of the present invention, the stroke volume (SV) is calculated for each individual while they are wearing the Biostrip on the chest, and asked to stay still for 60 secs, while a VCHE analysis is carried out. The User is then asked to exercise for a specified amount of time (1-150 mins), conducting a particular, pre-defined exercise (walking/running/cycling). After the completion of this exercise, another VCHE analysis is carried out, and the values described herein are estimated again. In this procedure, the stroke volume (SV) is calculated in the following manner:

SV=x₁*1/(PTT_{foot}*ΔPEP)+x₂*1/(PTT_{foot}*ΔPEP)²+x₃*1/(PTTₚₑₐₖ*ΔPEP)+x₄*1/(PTTₚₑₐₖ*ΔPEP)²; or

SV=ƒ(PTT_{foot},ΔPEP,PTTₚₑₐₖ,LVET,IVRT,IVCT,PEP,height,weight,BMI,age,gender);

Here, the constants xᵢ are derived from a population database, which assigns a distance for the individual being measured, to other subjects in the database, on the basis of height, weight, age, BMI, prior medical history and gender. If other information, such as blood test reports and/or echocardiograms and/or genetic tests are available, then these are also used to calculate the distance of the individual being measured to a similar subgroup (1-100 people) of the population. PTT_{foot} denotes the Pulse Transit Time between the R-peak from the ECG and the foot (beginning) of the PPG pulse wave, PTTₚₑₐₖ denotes the Pulse Transit Time between the R-peak from the ECG and the peak of the PPG pulse wave, and ƒ is a linear or non-linear function mapping the input parameters to the output variable. Once the distance function is derived, the correlation coefficients xᵢ or the function ƒ in the above equation are calculated by taking a weighted average of correlation coefficients previously calculated for patients who were simultaneously measured for SV using the Biostrip, and a standard Echocardiogram.

According to various embodiments of the current invention, the accelerometer on the Biostrip is used to measure vibrations of the sternum, above the aorta, when the Biostrip is stuck on the sternum, and the MCU on board the Biostrip is used to record the signals acquired at 10 Hz-2 KHz range. These signals are then processed by applying a Fourier transform, and then creating a spectral density map, which shows the intensity and time duration of different vibrations recorded on the sternum.

According to various embodiments of the current invention, the spectral density map of the vibrations of the sternum, above the aorta, recorded as described in [0074]. The peak frequency and duration of each frequency of vibrations, in the range of 10 Hz-2 KHz is recorded for every 1-10 second window. This measurement is then used to give a probabilistic estimate of a person is suffering from aortic stenosis, based on the following equation:

Aortic Stenosis Risk(ASR)=x₁*(Peak_Frequency−y₁)/y₁+x₂*(Peak_Frequency−190)*(Duration−y₂)/y₂; or

ASR=ƒ(Peak_Frequency,Duration,PEP,LVET,IVRT,IVCT,PWV,age,height,weight,BMI);

Here, y₁ is the peak frequency above which the risk of aortic stenosis has been found to be more common, and this value is set to 190 Hz in the initial instance. Peak_Frequency is also measured in Hz. Here y₂ is the duration (measured for every 1 sec window) for which the risk of high frequency vibrations is known to be more strongly correlated with aortic stenosis, and is set to 90 Hz in the first instance. Here x₁ and x₂ are constants which are chosen for each individual on the basis of their height, weight, BMI, age, gender and prior medical history, f is a linear or non-linear function that maps the input parameters to the output, and the final value of Aortic Stenosis Risk (ASR) is expressed as a percentage.

According to some embodiments of the present invention, the system for cardiovascular health monitoring can send an alert through the gateway device and/or through the vibration motor and/or the LEDs and/or an electronic display on the Biostrip, when the ASR, as recorded by the procedure described herein crosses a certain pre-defined threshold.

According to some embodiments of the present invention, the system for cardiovascular health monitoring is configured in such a way that the ECG and SCG measured on the chest with a Biostrip are combined with PPG signals measured on a User's fingers from the LED sensor on a smartphone, and the internal clock of the Biostrip is synced with the internal clock of the smartphone, and the signals from the Biostrip to an application running on the smartphone, which combines the data from the Biostrip and the internal PPG sensor on the smartphone, to arrive at values for PWV_{foot} and PWVₚₑₐₖ, which are used to estimate values for peripheral SBP and DBP, as well as other parameters of cardiovascular health, which depend upon the PWV, such as LVEF, or risk of Ischemia and atherosclerosis.

According to some embodiments of the present invention, the system for cardiovascular health monitoring is configured in such a way that the ECG and SCG measured on the chest with a Biostrip are combined with PPG signals measured on a User's wrist from a smartwatch, and the internal clock of the Biostrip is synced with the internal clock of the smartwatch, and the signals from the Biostrip to the smartwatch, which combines the data from the Biostrip and the internal PPG sensor, to arrive at values for PWV_{foot} and PWVₚₑₐₖ, which are used to estimate values for SBP and DBP, as well as other parameters of cardiovascular health, which depend upon the PWV.

In various embodiments of the current invention, the Biostrip device is configured to measure ECG and SCG from a Biostrip device stuck on the sternum near the aortic arch. The Biostrip device is further configured to estimate the Pulse Transit Time between the opening of the aortic valve, and the time-point at which the pressure pulse hits the aortic arch, by calculating the time-gap between the AO peak on the Z-axis plot of the SCG, and the sharp peak immediately after that, on the Y-axis of the SCG, which denotes the time of arrival of the pressure pulse, at the aortic arch (denoted by SCG_{YZ}). The system may be further configured to compute a value for PWV in the aorta, from the value of SCG_{YZ}, which is used to calculate central (aortic) systolic and diastolic blood pressure, and/or LVEF.

According to some embodiments of the present invention, the system is configured for the user or the caregiver to book appointments with a concerned doctor at a given hospital or clinic, and also to conduct an ‘Online Consultation’, by way of an audio/video VOIP call, or a regular phone call, or some other kind of communication protocol over the web.

During an online consultation, the system is also configured for the concerned personnel to view certain parts of the user's data, and to make episodic measurements from the Biostrip, through instructions delivered to the patient, and thereafter to view the data in real-time through a web application. In this manner, the system facilitates a ‘Virtual Examination’.

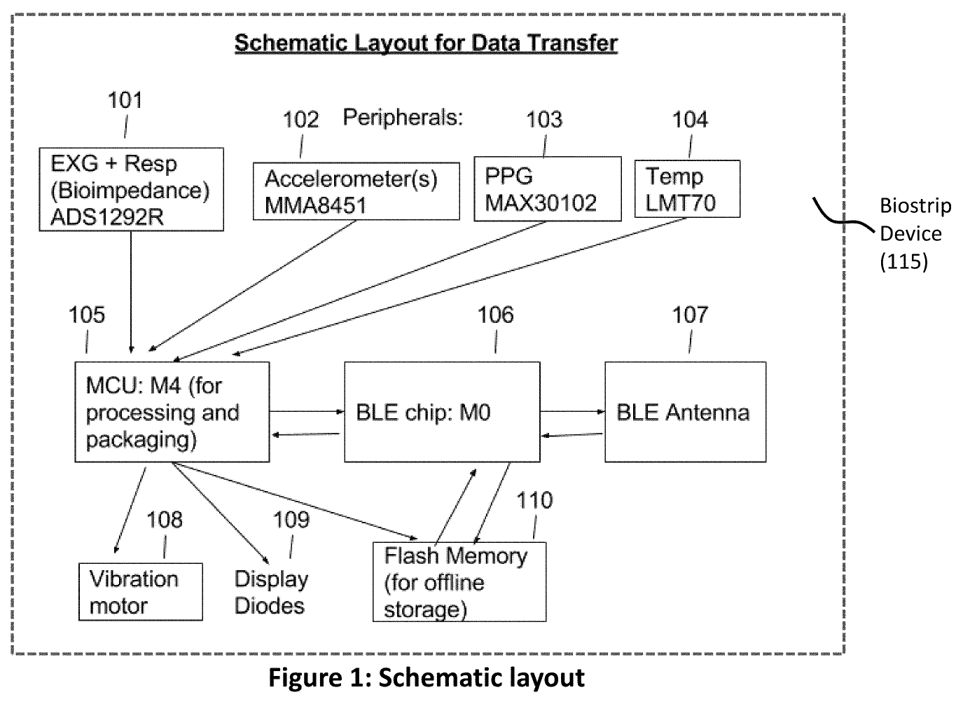

FIG. 1 shows a schematic layout of the Biostrip Device (115) and shows a plurality of sensors including an electrical sensor (101), accelerometer(s) (102), PPG sensor (103), Temperature sensor (104), that record data, and send the data to a microcontroller or microprocessor or another computing device (105) on the Biostrip.

As shown in FIG. 1, the computing device (105) receives outputs from the sensors (101, 102, 103, 104). The computing device (105) is configured to process this data as described elsewhere herein, and then send the data and/or results of the processing for transmission to the BLE chip (106). The wireless transmission module or BLE chip (106) then sends this data to the BLE antenna (107), which then communicates the data to other devices.

FIG. 1 also shows that in certain instances, on the Biostrip device includes a memory chip (110). Memory chip (110) is configured to store data received from the sensors (101, 102, 103, 104), and/or the output of microprocessor (105). The stored data may later be transmitted to another device. The Biostrip device also includes status diodes (109) which indicate different states of the device, and a vibration motor (108), which can alert the user when configured to do so, either by the MCU (110) or upon receiving an instruction from the smartphone/smartwatch (116).

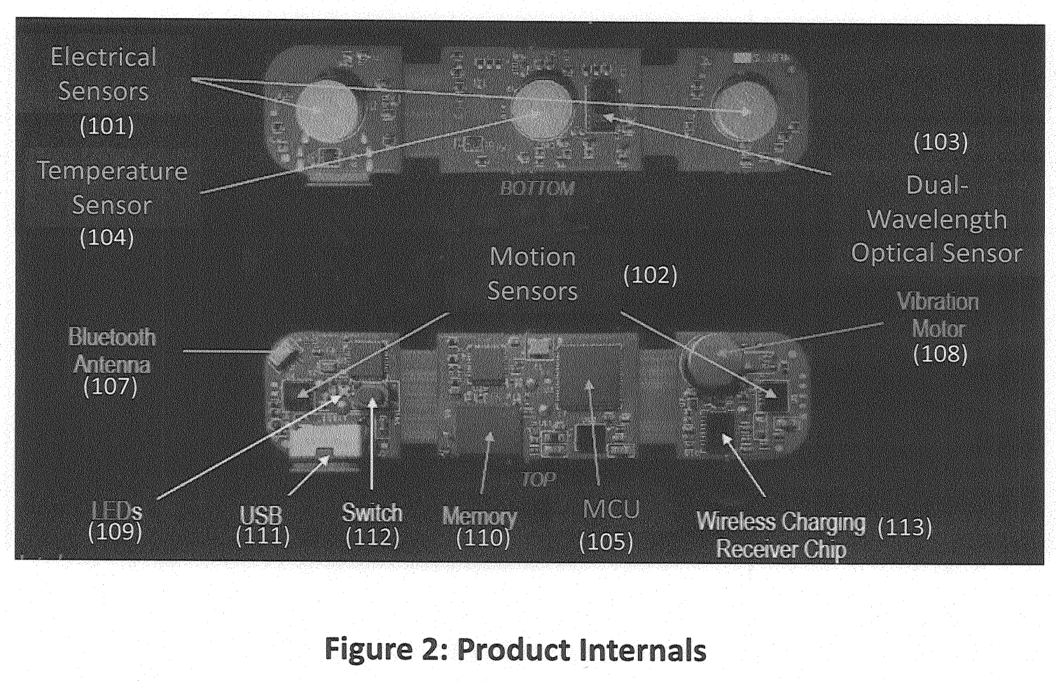

FIG. 2 shows a picture of the internal components of the Biostrip, in the actual configuration, as used in the product, according to various embodiments. The picture on the top shows the lower side of the PCBs, comprising the electrical sensors (101), the optical sensors (102), and the temperature sensor (104). The picture on the bottom shows the upper side of the PCBs, comprising of the accelerometers (102), the LEDs (109), the Bluetooth antenna (107), the USB port (111), the switch (112), the memory chip (110), the processor (105), the vibration motor (108) and the wireless charging receiver chip (113).

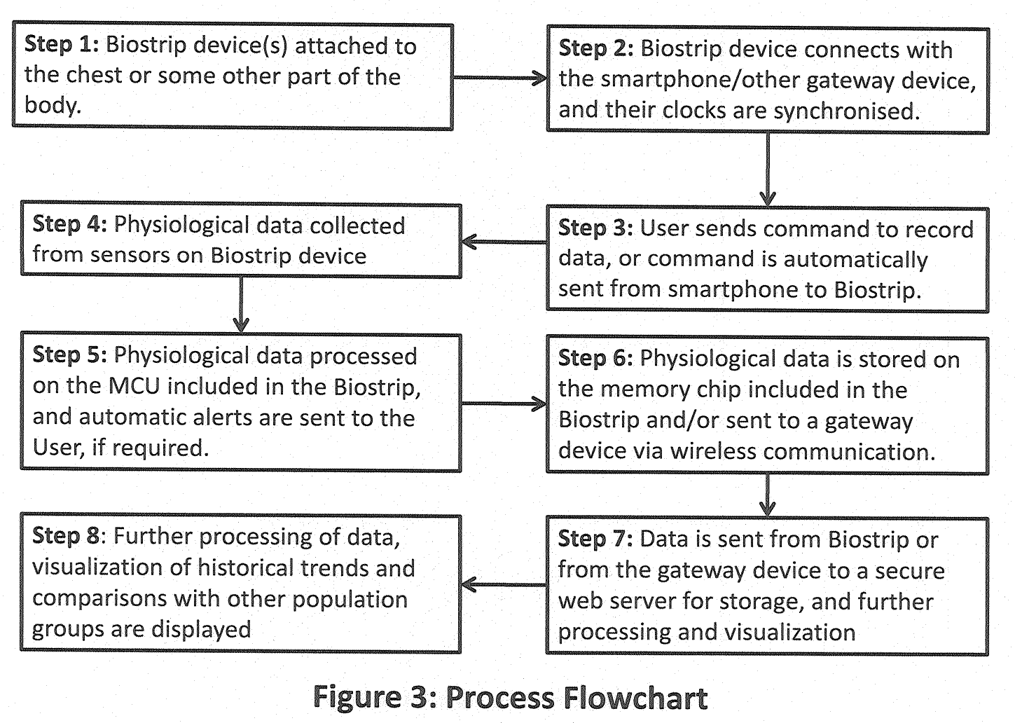

FIG. 3 shows a process flowchart for how the Biostrip and accompanying application function, in various embodiments. In Step 1, one or more Biostrip devices (115) are attached to some parts of the User's body. In Step 2, the Biostrip devices (115) connect with the smartphone or gateway device (116). In Step 3, the User or the User sends a command from the smartphone (116) to the Biostrip devices (115), to record physiological data from the User's body. In Step 4, physiological data is recorded by the Biostrips from sensors (101, 102, 103, 104) on the device. In Step 5, the data collected is processed by the MCU (105) on the Biostrip device (115). In Step 6, this processed data is stored on a memory chip (110) on the device, or sent to the smartphone (116) using some wireless communication protocol. In Step 7, this data is also sent from the Biostrip (115) or smartphone (116) to a secure storage location on the Cloud (117). In Step 8, the data is optionally further processed, and other insights are derived by processes running on the web server, as described elsewhere herein.

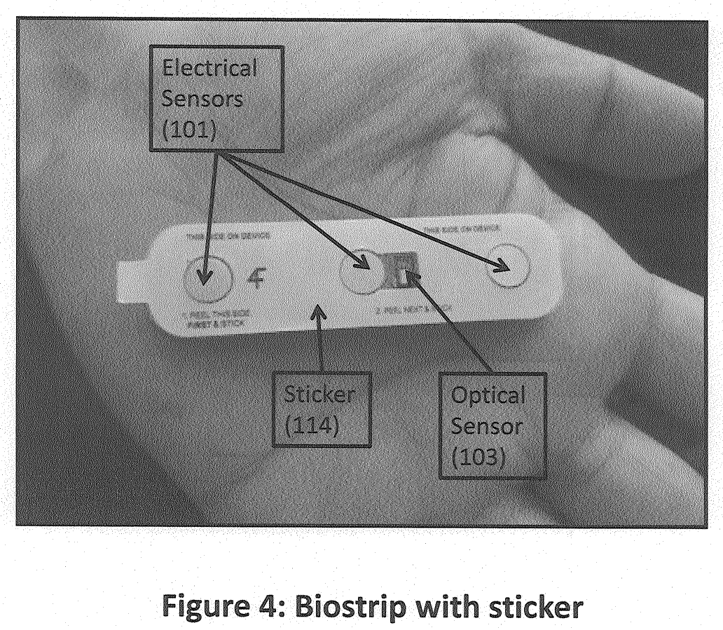

FIG. 4 shows a picture of the Biostrip (115) coupled with the double-sided sticker (114), one side of which adheres to the Biostrip (115), and the other side adheres to the skin of a User, when the Biostrip is worn by a User. The sticker (114) also contains cut-out holes for the electrical sensors (101) and the PPG sensor (103), so that the sensors (101,103) can be in direct contact with the skin.

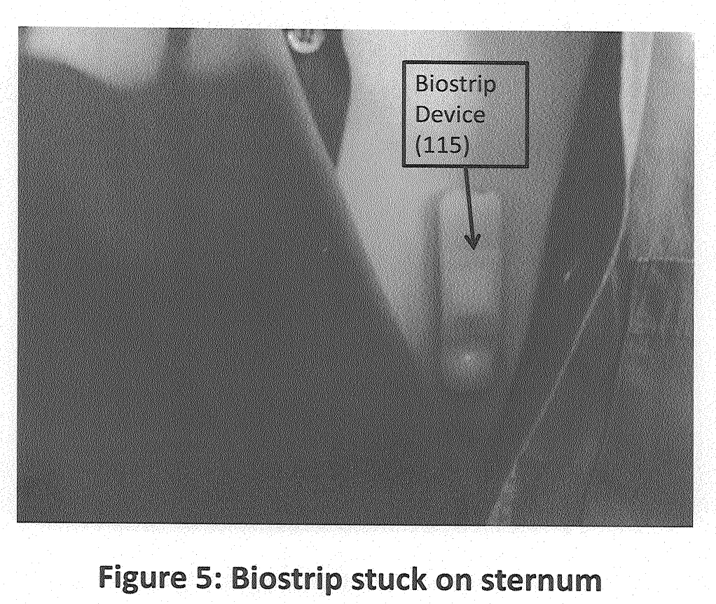

FIG. 5 shows a picture of the Biostrip device (115) worn by a User, in various embodiments, stuck on the User's sternum.

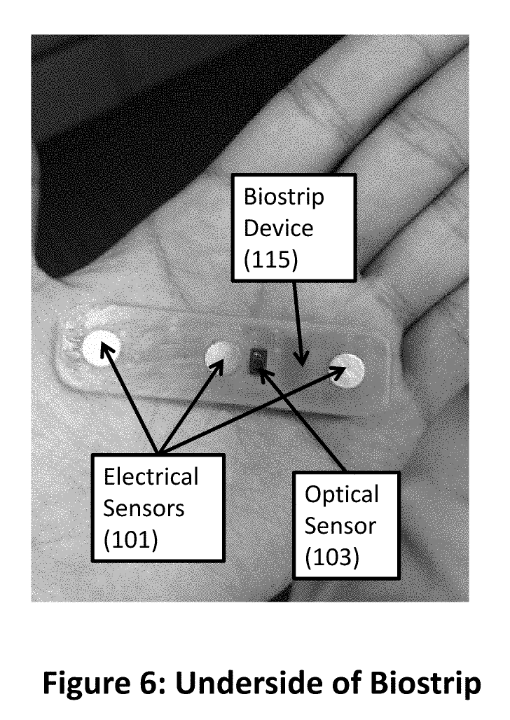

FIG. 6 shows the underside of the encased Biostrip, including three electrodes that together comprise the electrical sensors (101), the optical sensor (103), which generates and collects optical signals. These, and other components together make up the Biostrip device (115).

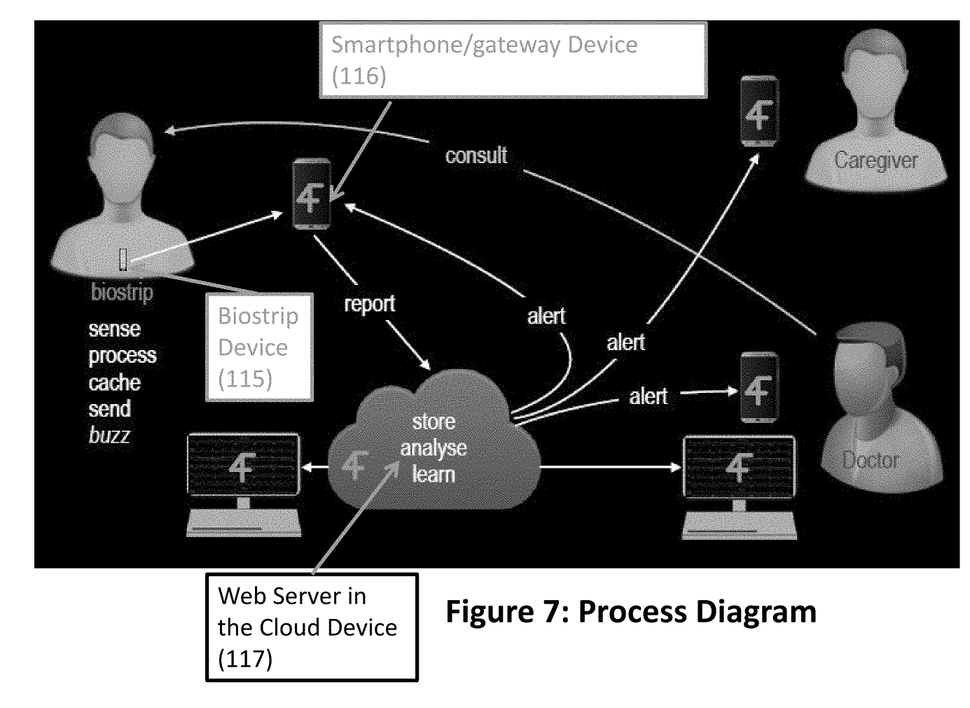

FIG. 7 shows various embodiments of the health monitoring system, comprising of the Biostrip device (115), which sends the data collected from the User to the smartphone or gateway device (116). The smartphone (116) then sends the data to the web server (117), where the data is processed, stored and/or analyzed in greater detail. Thereafter, the system can send alerts to the User, caregiver and/or Doctor, in various embodiments.

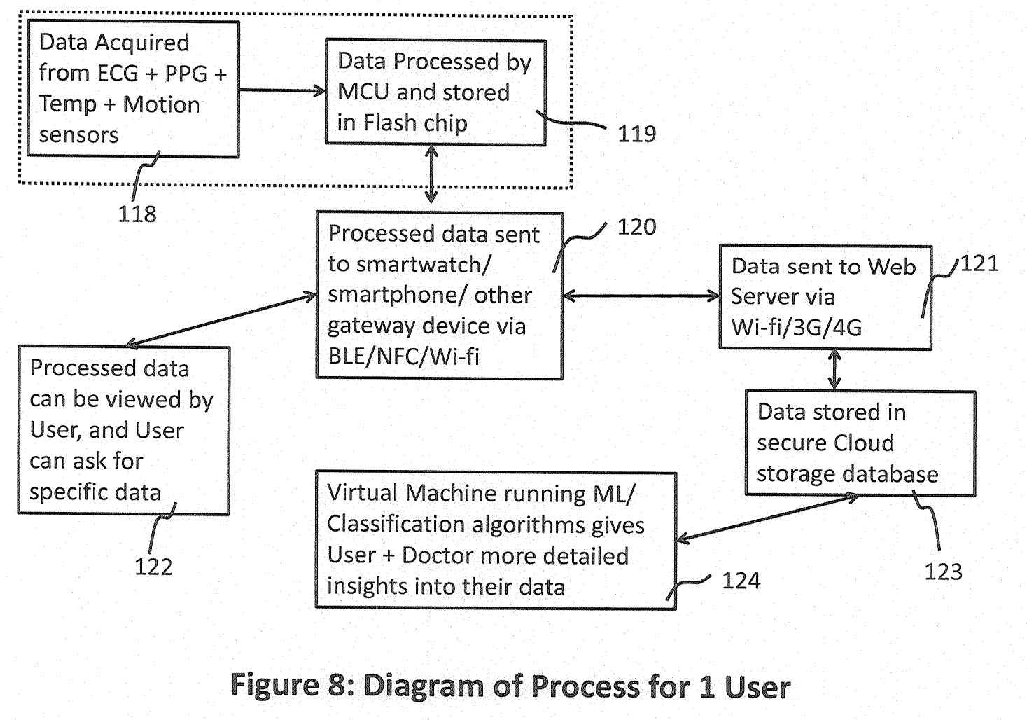

FIG. 8 illustrates various embodiments of the process of monitoring a single patient. In this embodiment, the system collects (118) the data from one or more Biostrip devices (115), and then processes (119) the data on the MCU (110) on board the Biostrip device (115). The data, after is it processed by the MCU (110), is sent (120) to the smartphone/smartwatch (116) or other gateway device via a wireless communication protocol. From the smartphone/smartwatch (116), the data can be sent (122) to the User, or it can be sent (121) to the web server via wifi or some other wireless communication protocol. From the web server, the data can be sent (123) to a secure Cloud database for storage. The data stored in the Cloud database is then accessed (124) by Machine Learning algorithms that can access this data and give the User or a Doctor more detailed insights into the data, and also push these insights into the Cloud database.

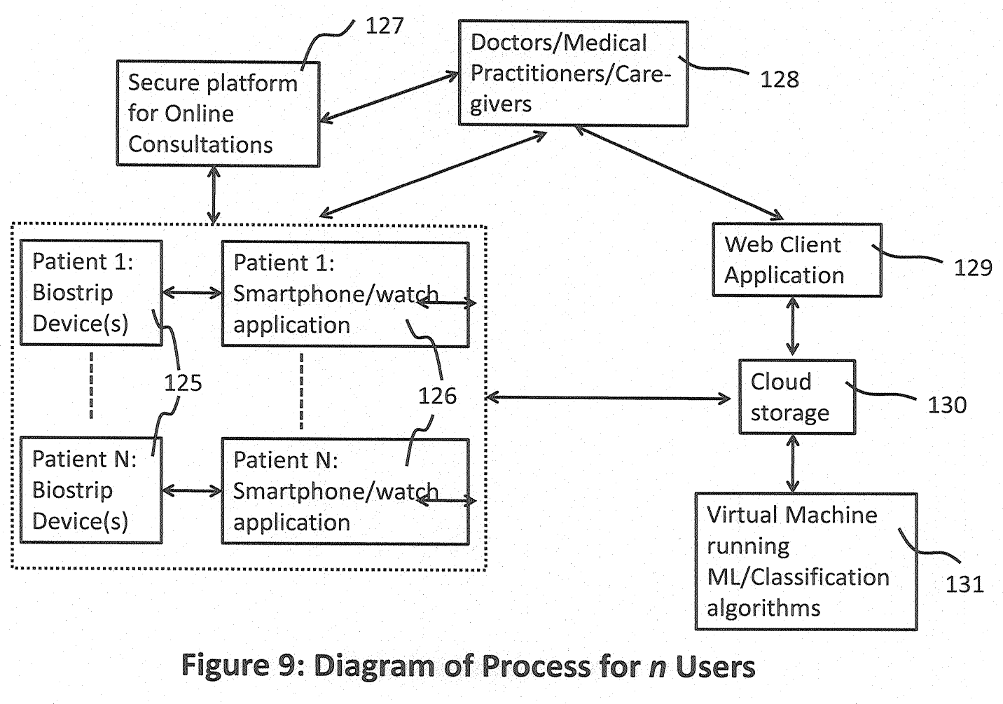

FIG. 9 shows a block diagram for the collection of physiological data by multiple Biostrips for multiple Users (125), in various embodiments. The data collected (125) from multiple Users and Biostrips, is sent (126) to the smartphones of the respective Users. From the smartphones (116), the data can be sent (127) to a secure platform for online consultation, which can then be accessed (128) by a Doctor/Caregiver, or be sent (129) to a web client application, or be sent (130) to the Cloud for storage purposes. From the Cloud storage (130), the data can be accessed (129) through a web client application. The data can also be accessed (131) by Machine Learning algorithms that can derive insights from this data.

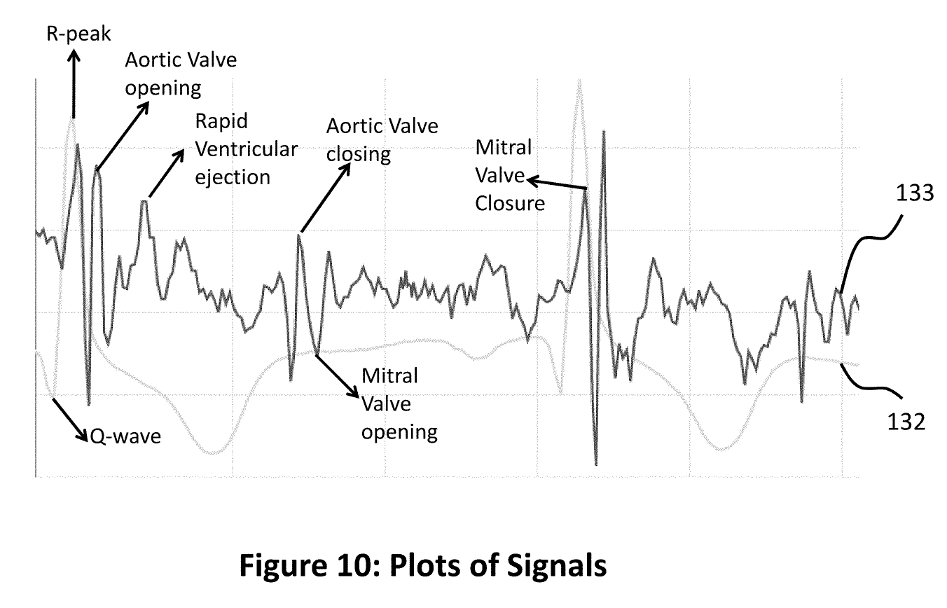

FIG. 10 shows an example of the signals captured on the Biostrip device (115), comprising of the ECG signal (132) and the SCG signal (133), which together denote several events of the cardiac cycle, including the P, Q, R, S and T complexes from the ECG, and the AO, AC, MO, MC, RF and RE events from the SCG. These are together used to calculate the cardiac time intervals discussed herein.

The foregoing description of the specific embodiments will so fully reveal the general nature of the embodiments herein that others can, by applying current knowledge, readily modify and/or adapt for various applications such as specific embodiments without departing from the generic concept, and, therefore, such adaptations and modifications should and are intended to be comprehended within the meaning and range of equivalents of the disclosed embodiments. It is to be understood that the phraseology or terminology employed herein is for the purpose of description and not of limitation. Therefore, while the embodiments herein have been described in terms of preferred embodiments, those skilled in the art will recognize that the embodiments herein can be practiced with modifications. However, all such modifications are deemed to be within the scope of the claims.

What is claimed is:

1. A wearable device comprising:
a) one or more PCBs;
b) a plurality of physiological sensors, including but not limited to at least two of: an ECG sensor, a Skin Impedance sensor, a PPG sensor, an Accelerometer and temperature sensor;
c) a computing device configured to record data from a subset of the sensors;
d) a double sided sticker configured to stick on a first side to the wearable device, and on a second side to skin of a user;

2. The device of claim 1, further including a wireless communication chip configured for the wearable device to connect wirelessly to a gateway device, and to send data to such gateway device through a wireless communication protocol.

3. The device of claim 1, wherein the physiological sensors are configured to record ECG, PPG or SCG waveforms, when the wearable device is stuck on a chest of the User.

4. The device of claim 1, wherein the double-sided sticker includes a plurality of cut-out holes, each of the cut-out holes adapted to receive at least one sensor, the cut-out holes further adapted to allow the at least one sensor to be in direct contact with the skin.

5. The device of claim 1, further including an Ultrasound transducer configured for recording an Ultrasound signal.

6. The device of claim 1, wherein the one of the PCBs further includes one or more of the following:
a USB port;
one or more LEDs;
a thermoelectric or photoelectric panel for harvesting energy from the body heat, or from light or heat in the environment;
an electronic display; and
a wireless charging coil.

7. The device of claim 1, wherein the computing device is further configured to measure ECG, SCG or PPG waveforms in parallel, when stuck on a chest of the User, and further configured to perform calculations that de-noise each of the waveforms, and derive parameters related to the cardiovascular health of the individual, the parameters including at least one of the following: Heart Rate, Respiration Rate, Arrhythmias, Heart murmurs, Pulse Wave Velocity, Blood Pressure, Respiratory Sinus Arrhythmia, Cardiac Time Intervals (PEP, LVET, IVRT, IVCT), Left Ventricular Ejection Fraction (LVEF), Cardiac Output and Stroke volume.

8. The device of claim 7, wherein the computing device is further configured to record accelerometer data, and to calculate a value of Energy spent and Power consumed by the User in different time intervals and to use the Energy spent to calculate a change in parameters related to the cardiovascular health due to exercise.

9. The device of claim 3, wherein the computing device is further configured to record ECG data from the sensors, and to record SCG data collected from the accelerometers, and to automatically detect cardiac events including one or more of the following: Heart murmurs, Aortic valve opening (AO), Mitral valve opening (MO), Aortic valve closure (AC), Mitral valve closure (MC), Rapid Ejection (RE), Rapid Filling (RF) and Atrial Systole (AS).

10. The device of claim 1, wherein the computing device is configured to calculate a spectral density function applied to vibrations of the sternum, above the aorta, measured at 5-2000 Hz, and to measure peak frequencies, and durations of the peak frequencies.

11. The device of claim 3, wherein the computing device is further configured to combine information from an ECG sensor, an SCG sensor, and a PPG sensor to calculate parameters including one or more of the following: Heart Rate, Pre-ejection period (PEP), left ventricular ejection time (LVET), QS1, QS2, S1S2, PR-interval, QRS duration, Systolic Time, Diastolic Time, PTT_{foot}, PTTₚₑₐₖ, Electro-mechanical Activation time (R-peak to MC), Isovolumetric Relaxation Time (IVRT), Isovolumetric Contraction Time (IVCT), central (aortic) Systolic and Diastolic Blood Pressure, and LVEF.

12. The device of claim 3, wherein the computing device is further configured to record data obtained from ECG and SCG sensors, and to calculate values of one or more of the following: PEP, LVET, IVRT, IVCT, PQ-interval, ST-interval, Amp (AOₚₑₐₖ): when placed on a sternum of the User, and further configured to calculate a value of central (aortic) Systolic and Diastolic Blood Pressure, and LVEF, using a calibration based approach or using a population-based regression model that includes information on the one or more of the following: LVEF, aortic blood pressure, SBP, DBP, LVEF and height, weight, gender and prior medical conditions of the user.

13. A system for monitoring cardiovascular health of a user, comprising:
a) One or more wearable devices, each constructed on one or more PCBs, the wearable devices each including one or more of the following physiological sensors: ECG sensor, PPG sensor and Accelerometer;
b) A double-sided sticker capable of affixing the wearable device on the User;
c) A gateway device configured to obtain signals from the wearable device;
d) A web server wherein the sensors are configured to store data collected from (a) or (c) above, and the web server is configured to calculate parameters related to the cardiovascular health of the user.

14. The system according to claim 13, configured to measure the values of one or more of the following: PEP, LVET, HR, PWV, amplitude of PPG peak and amplitude of AO peak.

15. The system according to claim 14, wherein the sensors are further configured to measure a Cardiac Health Index (CHI) of the form:
CHI=ƒ(PEP,LVET,amp(AO),amp(PPG),IHR,PWV,height,weight,BMI,age,blood profile,genetic profile),
Where ƒ is a linear or non-linear function that maps the input parameters to the output CHI.

16. The system according to claim 13, wherein the Biostrip device or the smartphone or the web server, is configured to calculate a value for Left Ventricular Ejection fraction in the form of:
LVEF=ƒ(PEP,LVET,amp(AO),amp(PPG),IHR,PWV,IVRT,IVCT,height,weight,age,BMI,blood profile,genetic profile);
where, ƒ is a linear or non-linear function which maps the input parameters to the output LVEF, optionally using a population database.

17. The system according to claim 13, wherein the Biostrip device or smartphone or web server is configured to compute cardiovascular health variables including one or more of the following: HR, LVET, PEP, MPI, LVEF, CHI, Cardiac Output, Stroke Volume, Arrhythmias (type and duration), and heart murmurs.

18. The system according to claim 17, wherein the web server is further configured to calculate a confidence interval corresponding to one or more of the following: HR, LVET, PEP, MPI, LVEF, CHI, Cardiac Output, Stroke Volume, Arrhythmias, and heart murmurs: on the basis of a comparison between the raw sample, histogram or Fourier Transform of the measured signal, and a corresponding clean sample signal which is pre-loaded in the memory of the Biostrip device or gateway device or web server.

19. The system according to claim 17, wherein the Biostrip device is further configured to send alerts to the User via a Vibration motor, LEDs, a display or an audio speaker on the Biostrip device, and wherein the smartphone or the web server is further configured to send alerts to another individual or institution via wireless communication, when one or more of the following parameters: HR, LVET, PEP, MPI, LVEF, CHI, Cardiac Output, Stroke Volume, Arrhythmias, and heart murmurs: are found to lie outside a certain pre-defined range.

20. A system according to claim 17, wherein the Biostrip device is configured to calculate values for one or more of the following: LVEF, Amp(AO), Amp(RF) or PEP: and wherein the Biostrip device is further configured to use the calculated values measured before and after a pre-specified exercise regimen, to calculate a value of “Cardiac Health Risk” (CHR), that represents a percentage probability of a Major Cardiovascular Event (MACE) in the next 1 year, value of CHR being calculated in the following manner:
CHR=ƒ(Amp(AO_{before},Amp(AO_{after}),Amp(RF_{after}),PEP_{after},PEP_{before},LVET_{before},LVET_{after},LVEF_{before},LVEF_{after},BMI,height,weight,age,gender,blood profile,genetic profile);
where ƒ is a linear or non-linear function that maps the input variables to the output CHR, optionally using a pre-specified population database.

21. The system according to claim 13, comprising 2-6 of the Biostrip devices together the user, stuck at different locations on the chest of the user, and configured to mimic a 2-6 Lead Holter monitor and to record and transmit ECG data.

22. A system, according to claim 21, that uses the method of co-locating sharp taps/spikes on the accelerometer, or the R-peak of the ECG to synchronize the internal clocks of multiple Biostrips with each other, or the internal clocks of one or more Biostrips with the internal clock of the smartphone/smartwatch.

23. A system according to claim 13, wherein the Biostrip device and the smartphone/smartwatch are configured to calculate one or more of the following: PAT_{foot}, PATₚₑₐₖ, PAT_{50%}, PEP, systolic and diastolic Blood Pressure: from a combination of: ECG and/or SCG measured on the Biostrip device stuck on the chest, and the PPG measured on the smartphone or smartwatch using an in-built LED, when the finger is placed on it.

24. A system, according to claim 13, wherein the Biostrip device is configured to measure ECG and SCG from a Biostrip device stuck on the sternum near the aortic arch, further configured to estimate the Pulse Transit Time between the opening of the aortic valve, and the time-point at which the pressure pulse hits the aortic arch, by calculating the time-gap between the AO peak on the Z-axis plot of the SCG, and the sharp peak immediately after that, on the Y-axis of the SCG, which denotes the time of arrival of the pressure pulse, at the aortic arch (denoted by SCG_{YZ}). The Biostrip device or smartphone or web server further configured to compute a value for PWV in the aorta, from the value of SCG_{YZ}, which is used to further calculate central (aortic) systolic and diastolic blood pressure, and/or LVEF.

25. A system, according to claim 13, wherein the web server is configured to present the user and the authorized third-party with periodic report of the cardiovascular health of the User recorded during different activities, compared with the cardiac health parameters of other subjects in a similar population group, at different times of the day, and further to give recommendations to the user or care-giver or a doctor for different ways of controlling different cardiovascular health parameters more effectively at different times of the day.
